(19) United States
(12) Patent Application Publication
(10) Pub. No.: US 2010/0057077 A1
(43) Pub. Date: Mar. 4, 2010

(54) FEP PRE-CURVED DISTAL TIP SPHINCTEROTOME

(75) Inventor: Richard W. Ducharme, Winston-Salem, NC (US)

Correspondence Address: BRINKS HOFER GILSON & LIONE/CHICAGO/COOK, PO BOX 10395, CHICAGO, IL, 60610, US

(73) Assignee: Wilson-Cook Medical Inc., Winston-Salem, NC (US)

(21) Appl. No.: 12/202,900

(22) Filed: Sep. 2, 2008

(51) Int. Cl. A61B 18/12

(52) U.S. Cl. 606/39

(57) ABSTRACT

A method and device for incising tissue within the gastrointestinal tract is described. The device is an electrosurgical sphincterotome cutting device. The sphinctertome includes a pre-curved, heat set FEP distal tip. An electrically conductive cutting wire is positioned along the pre-curved distal tip. Manipulating a control handle tightens the cutting wire and incises and cauterizes target tissue. The curvature of the distal tip allows the sphincterotome to orient and steer itself towards a patient's sphincter as it emerges from an accessory channel of an endoscope.

### TECHNICAL FIELD

The invention generally relates to a sphincterotome having a pre-curved distal tip that provides controlled cutting and orientation during, for example, the cutting of a patient's sphincter.

### BACKGROUND

Gastrointestinal endoscopy is commonly used to gain access to the digestive tract for the purpose of incising and cauterizing tissue. Many common endoscopy procedures exist for achieving this purpose.

Endoscopic sphincterotomy is a specific procedure in which a sphincterotome is used in combination with an endoscope to surgically cut a patient's sphincter. As one example, the sphincterotome may be used to partially cut open the duodenum at the Papilla of Vater to access the common bile duct and remove bile duct stones which form an obstruction therewithin. Conventional sphincterotomes utilized in this technique can create major complications, including bleeding, pancreatitis, perforation, and cholangitis. Bleeding is a common complication which arises when the retroduodenal artery is inadvertently cut. This inadvertent cut of the artery may often be caused by a lack of cutting control of the sphincterotome. As a result, practitioners must be able to properly orient the cutting wire of the sphincterotome at the optimal location for accessing the sphincter or papilla of a patient.

Inducing a curve or bend in the distal end of the sphincterotome may facilitate the proper orientation of the device. This is typically accomplished by placing a shaping wire in the wire guide lumen at the distal end of the sphincterotome. The shaping wire tends to curve, at least temporarily, the distal end of the device. However, because of the materials typically used to form the shaft of the distal end of the sphincterotome, the distal end of the device may begin to straighten as soon as the shaping wire is removed. Thus, it is often necessary to re-insert the shaping wire to re-curve the distal end of the device, thereby increasing the duration of the procedure. In addition, the distal end of the sphincterotome tends to straighten as the device is advanced through the endoscope towards the patient's papilla. As a result, it may be difficult to cannulate the biliary or pancreatic ducts and achieve the desired cutting orientation. This can also increase procedure time and may result in the improper cutting of the papilla. As a result, conventional sphincterotomes are prone to the problem of achieving adequate orientation. The inability to achieve adequate orientation may lead to uncontrolled cutting and cauterization. The use of a shaping wire may also interfere with the ability to pre-load a wire guide or other elongate device into the sphincterotome, thereby further increasing the complexity and duration of the procedure.

In view of these drawbacks of current technology, there is an unmet need for incision devices that can controllably access, cut and cauterize tissue without inducing significant patient trauma.

### SUMMARY

Accordingly, an electrosurgical cutting device is provided that resolves or improves upon one or more of the above-described drawbacks.

In a first aspect, an electrosurgical cutting device is provided. The device comprises a tubular member comprising a proximal end and a distal end. The distal end comprises a heat-set, pre-curved distal tip formed from fluorinated ethylene propylene (FEP). An electrically conductive cutting element is located along the distal end of the tubular member. The cutting element is connected to an electrical conductor extending within a lumen. The cutting element extends exteriorly of the tubular member along an inner radius of curvature of the distal tip. The cutting element is moveable within a cutting plane. The arrangement insures that the distal tip of the tubular member will maintain the desired cutting orientation as it emerges from a distal end of a working channel of an endoscope so as to position the cutting element within the desired cutting plane. A wire guide may be pre-loaded through a wire guide lumen of the electrosurgical cutting device with a distal end of the wire guide extending beyond the distal end of the tubular member.

In a second aspect, a method of fabricating an electrosurgical cutting device is provided. A proximal end of an electrical conductor is attached to an electrical connector of a handle. The handle is affixed to a substantially linear tubular member formed from fluorinated ethylene propylene (FEP). An electrical conductor is threaded through a lumen. A distal free end of the electrical conductor is passed through a proximal luminal opening of the tubular member along the distal tip and outward of the lumen to form a cutting element. A distal free end of the cutting element is reinserted through a distal luminal opening of the tubular member into the lumen to secure the distal end within the lumen. A distal end of the straight tubular member is heat set into a curved distal tip that conforms to a shape of a scaffolding structure having a corresponding curved distal end.

### BRIEF DESCRIPTION OF THE DRAWINGS

Embodiments will now be described by way of example with reference to the accompanying drawings, in which:

FIG. 1 is a perspective view of a sphincterotome and loop tip wire guide according to an embodiment;

FIG. 2 is a cross sectional view of FIG. 1 taken along cutting line III-III showing a wire guide extending through one of the lumens of the sphincterotome and an electrical conductor extending through the other lumen;

FIG. 3 is a side view of the distal tip of the sphincterotome with cutting wire in the 12 o'clock orientation relative to the papilla as the sphincterotome emerges from a distal end of an accessory channel of an endoscope, the distal tip being navigated over the loop tip wire guide;

FIG. 4 is a perspective view of the distal tip of the sphincterotome in a desired cutting plane configuration;

FIG. 5 is a view of the papilla with the cutter oriented in the 12 o'clock position relative to the papilla;

FIG. 6 is a partial view of the pre-curved sphincterotome being used to perform an endoscopic sphincterotomy procedure;

FIG. 7 is a plan view of a packaging tray used for shipping, handling, and storing the pre-curved sphincterotome with pre-loaded wire guide therein; and

FIGS. 8-10 are side views of the cutting wire of the sphincterotome at various locations along the pre-curved distal tip.

### DETAILED DESCRIPTION OF THE PREFERRED EMBODIMENTS

The embodiments are described with reference to the drawings in which like elements are referred to by like numerals. The relationship and functioning of the various elements of the embodiments are better understood by the following detailed description. However, the embodiments as described below are by way of example only, and the invention is not limited to the embodiments illustrated in the drawings. It should also be understood that the drawings are not to scale and in certain instances details have been omitted, which are not necessary for an understanding of the embodiments, such as conventional details of fabrication and assembly.

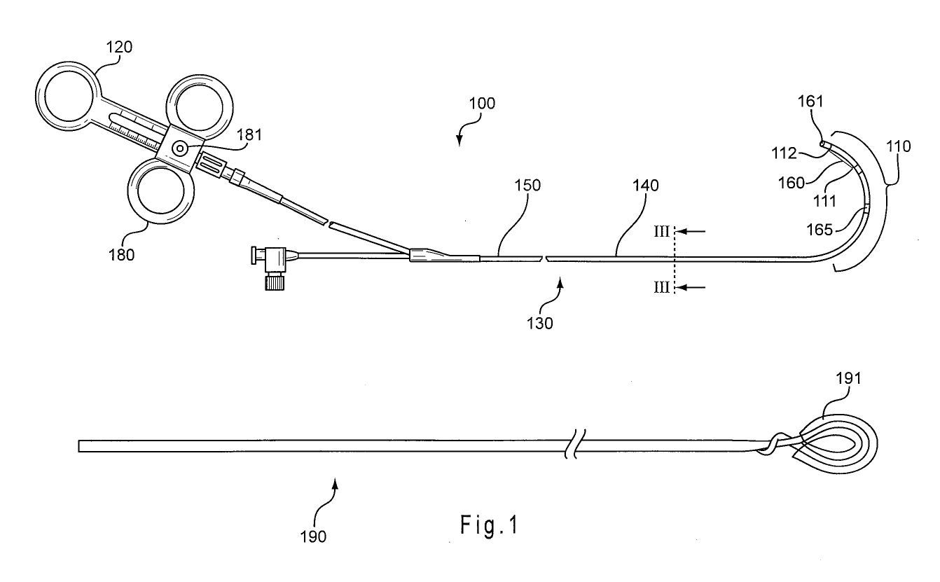

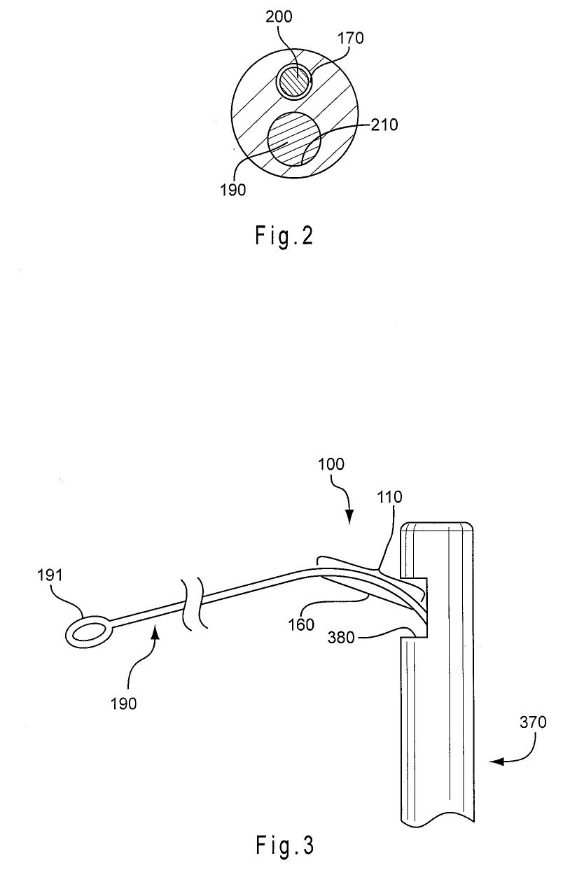

An exemplary sphincterotome is shown in FIG. 1. FIG. 1 is a perspective view of a sphincterotome 100 and a separate loop tip wire guide 190 (which has been enlarged for clarity). The sphincterotome 100 includes a tubular member 130 having a proximal region 150 and a distal region 140. The proximal region 140 includes a control handle 120. The control handle 120 comprises a movable hand portion 180 which may be drawn proximally to draw tension on a conductor wire 200 (FIG. 2) during an endoscopic sphincterotomy procedure. The distal region 140 includes a pre-curved distal tip 110. The term “pre-curved” as used herein refers to a distal tip of a fluorinated ethylene propylene (FEP) tubular member 130 that is heat set into a permanently curved configuration during fabrication. FIG. 1 shows the pre-curved distal tip 110 in its normal, relaxed state. At least the pre-curved distal tip 110 is formed from FEP. FEP is a thermoplastic which can be melt processed and shaped by application of pressure and heat. The polymeric chemical structure of FEP enables it to be readily shaped using conventional thermoplastic processing techniques, including injection, transfer, blow, and compression molding as well as screw extrusion. Thermoset materials, on the contrary, cannot be melt processed. PTFE is one of the most common thermoset materials used in medical devices because of its excellent chemical resistance. As a result, PTFE is typically used to manufacture the tubular shaft portion of conventional sphincterotomes. However, PTFE does not have a chemical structure that is amenable to melt processing because the chemical structure of PTFE includes strong C—F bonds in which the fluorine atoms are packed tightly in a spiral manner about the carbon backbone, which is what makes PTFE one of the most chemically resistant synthetic polymeric materials.

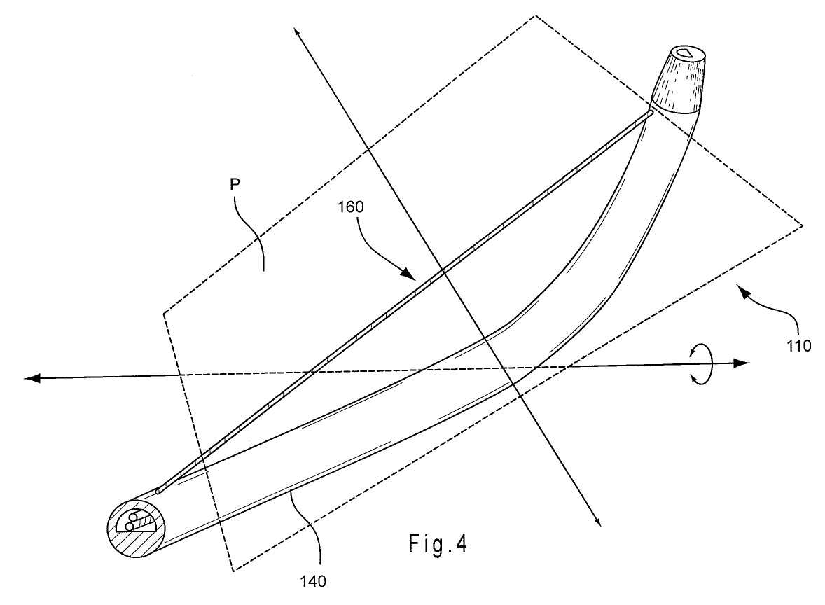

The FEP tubular member 130 may be extruded and thereafter a portion of the distal region 140 may be heat set into the pre-curved distal tip 110 having the desired cutting orientation as shown in FIG. 1. Generally speaking, during an endoscopic sphincterotomy procedure, as the pre-curved distal tip 110 emerges from the working channel 380 of an endoscope 370, it orients into the optimal 12 o'clock position (FIGS. 3, 5, and 6) thereby enhancing the ease of cannulation. The pre-curved distal tip 110 maintains its orientation with the cutting plane P (FIG. 4) which enables the cutting wire 160 to approach a patient's papilla 510 at the desired 12 o'clock orientation relative to the papilla 510.

Because the FEP pre-curved distal tip 110 is at the time of fabrication oriented into the desired curved orientation, shaped forming wires or other reinforcing means are not needed to form and maintain the distal tip 110 in the curved shape during shipping and storage prior to use. Accordingly, the absence of a need for a forming wire within the wire guide lumen 210 (FIG. 2) allows a loop tip wire guide 190 (FIG. 1) or other elongate medical device to be pre-loaded therethrough. FIG. 1 shows an exemplary loop tip wire guide 190 (not drawn to scale) that may be pre-loaded within the wire guide lumen 210 (FIG. 2) and positioned so as to extend the distal loop tip 191 beyond the distal edge 161 of pre-curved distal tip 110 of tubular member 130. This is in contrast to conventional sphincterotomes which cannot be pre-loaded with a loop tip wire guide. Therefore, the absence of the need for the forming wire in the sphincterotome 100 of the present invention allows a loop tip wire guide 190 to be pre-loaded within the sphincterotome 100. Other devices may also be pre-loaded within the lumen 210. As will be later described, the pre-loaded sphincterotome 100 may be packaged as shown in FIG. 7. The loop tip wire guide 190 unlike other elongate devices, such as a non-loop tip wire guide, presents the additional problem of being pre-loaded into sphincterotome 100 by advancing it at the proximal end of the sphincterotome 100 rather than through the distal end of the sphincterotome 100. Although it is possible to pre-load other medical devices by partially advancing them through the proximal end of the sphincterotome 100, the loop tip wire guide 190 cannot be advanced into the sphincterotome 100 due to the presence of a forming wire blocking the lumen 210.

Although FIG. 2 shows two lumens extending through tubular member 130, more than two lumens are also contemplated. For example, a third lumen may be dedicated for the passage of fluids or contrast fluid therethrough.

Preferably, FIG. 2 shows that the sphincterotome 100 comprises two lumens. Lumen 210 is adapted to receive a wire guide, including the loop tip wire guide 190 of FIG. 1. This lumen 210 may also be configured for the passage of fluids or contrast therethrough. Lumen 170 is adapted to receive an electrical conductor wire 200. Although the lumens 170 and 210 are shown with circular cross-sectional shapes, other lumen shapes are possible.

The electrical conductor wire 200 transmits current to the cutting wire 160. The conductor wire 200 is a wire extending through lumen 170 (FIG. 2) and is connected at its proximal end to electrical connector 181 (FIG. 1) to provide a high frequency electrical current to conductor 200 and cutting wire 160 as is well known to one of ordinary skill in the art. Conductor 200 protrudes outward of the wall of tubular member 130 at the distal tip 110 through first opening 111 to become cutting wire 160. The cutting wire 160 is bowed between the first opening 111 and the second opening 112 and is disposed outside of the wall of tubular member 130. The cutting wire 110 re-enters the wall of the tubular member 130 through second opening 112 and extends proximally through the lumen 170. Preferably, the conductor 200 and cutting wire 110 may be formed from a single wire. Alternatively, the cutting wire 110 and conductor 200 may be distinct components that may be connected to each other by soldering or other conventional means known in the art.

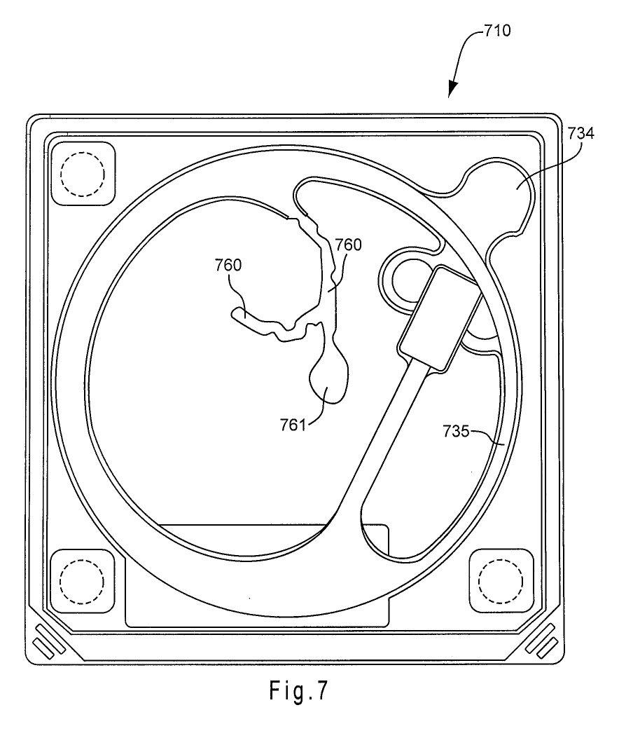

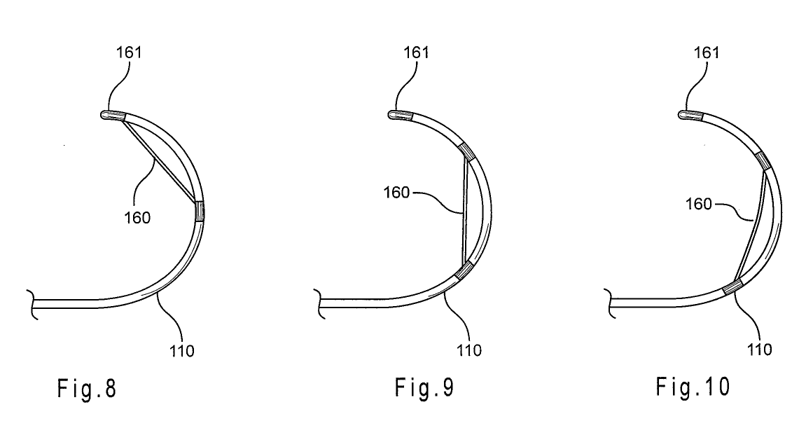

Although FIG. 1 shows the cutting wire 160 positioned near the distal end 161 of the distal tip 110, the cutting wire 160 may be positioned at different locations along the pre-curved distal tip 110. Additionally, the cutting wire 160 may be of various lengths by changing the proximal and distal openings 111 and 112. FIG. 8 is an enlarged view of FIG. 1, showing the cutting wire 160 positioned at the distal edge 161 of distal tip 110 along the inner radius of the pre-curved distal tip 110. FIGS. 9 and 10 show the cutting wire 160 positioned substantially along the center of the pre-curved distal tip 110. The primary difference between FIG. 9 and 10 is that the cutting wire 160 of FIG. 10 is longer than the cutting wire 160 of FIG. 9. As a result, the cutting wire 160 of FIG. 10 may be able to remove a larger amount of tissue or cut to a greater depth. The specific location of the cutting wire 160 along the distal tip 110 is dependent upon many factors, including the amount of tissue being cut and cauterized.

The proximal end of the conductor wire 200 is connected to the control handle 120 such that actuation of the handle assembly 120 partially retracts (i.e., pulls in a proximal direction) the conductor wire 200 and cutting wire 160 to exert a tension therealong. This causes the distal end of the cutting wire 160 to pull against the already pre-curved distal tip 110, thereby causing the distal tip 110 to bow inwards even more to further reduce the inner radius of the pre-curved distal tip 110. Electric current that passes through the conductor wire 200 from electrical connector 181 in the control handle 120 enables the cutting wire 160 to act as an electrosurgical cutting element that may be used to cut and cauterize tissue, such as the sphincter of Oddi.

The distal edge 161 of tubular member 130 may comprise a tapered shaped end. The tapered distal edge 161 may comprise a reduction in wall thickness of tubular member 130 and a reduction in outer diameter. Because the tapered distal edge 161 comprises rounded edges, it may mitigate trauma to a patient as the distal edge 161 is being navigated within a body lumen. The FEP distal edge 161 may be tapered under suitable heat and pressure and is relatively easier to shape compared to tips not formed from thermoplastics. For example, because tips made from PTFE are not readily melt processable, relatively higher pressures and temperatures are required to form a tapered tip. Such higher pressures and temperatures may likely translate into relatively more energy intensive and expensive process compared to distal tips formed from FEP.

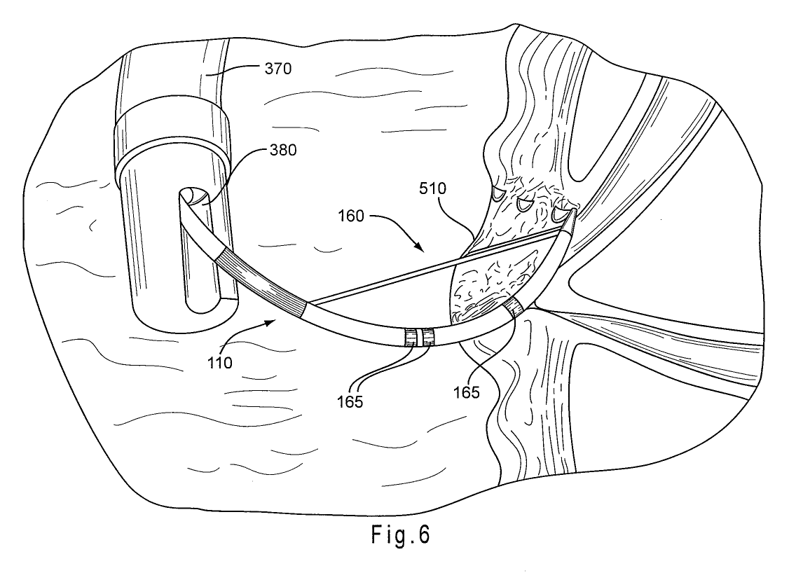

A radiopaque marker band 165 (FIG. 1 and FIG. 6) may be thermally bonded along the distal tip 110 to enable fluoroscopic visualization of the distal tip 110 as it is being maneuvered. The radiopacity of the distal tip 110 provides information to a physician regarding the location and orientation of the distal portion 110 in various body lumens that the sphincterotome 100 is being guided through. Conventional sphincterotomes formed from thermoset materials such as PTFE cannot be reheated so as to form a thermal bond with the radiopaque marker band 165 because PTFE is not a melt processable material. Reheating of PTFE results in reaching PTFE's thermal decomposition temperature before its melting point is obtained. As a result, PTFE cannot be melted and re-shaped after it is cured. PTFE sphincterotomes generally utilize a metallic band mechanically secured by, for example, crimping about the catheter shaft, in which no thermal bonding occurs. There may be a risk that the crimped metallic band is not effectively secured to the shaft and could detach from the tubular member. Accordingly, the ability for a FEP tubular member 130 to be thermally bonded with a radiopaque marker band 165 is advantageous.

Various techniques may be utilized to form the pre-curved distal tip 110. In one example, an internal curved mandrel may be utilized in which the mandrel is inserted into one of the lumens of the tubular member 130. Because FEP is soft at room temperature, the FEP tubular member 130, which is substantially a straight extruded tubing when initially formed, is flexible enough to accommodate the curved shape mandrel. The mandrel may be back loaded into wire guide lumen 210 from the distal edge 161 of tubular member 130. The length of mandrel may be the length of the resultant distal tip 110 (FIG. 1). Alternatively, the mandrel may be longer, having a straightened proximal portion. Having loaded the curved mandrel within one of the lumens of the FEP tubular member 130, the heat setting process may begin. The process variables for heat setting are generally temperature, time and pressure and may be adjusted as needed to create the necessary curved distal tip 110. Specifically the heat-setting temperature is sufficient for the FEP thermoplastic material to lose its crystallinity and become amorphous in structure such that that the FEP material becomes flowable, thereby conforming to the curved shape of the internal mandrel. In one embodiment, the heat setting procedure involves heating the FEP material to about (300-600)° F. for Up to about 15 minutes. Suitable ranges of temperatures, time and pressures appropriate for the heat-setting process may be readily determined by those skilled in the art. Having shaped at least a portion of the distal region 140 of the tubular member 130 into a curved distal tip 110, the resultant curved shape is quenched in a cool down cycle at a predetermined cooling rate. The precise cooling rate varies depending on numerous factors, including the desired crystallinity and amorphousness of the resultant FEP pre-curved distal tip 110. In one embodiment, the cool down cycle involves cooling the FEP material to about ambient temperature for about 5 minutes.

Alternatively, an external mandrel may be utilized in which the tubular member 130 is inserted into a passageway of the mandrel. Upon suitable heat and pressure for a given duration of time, a portion of the distal region 140 becomes heat set into a curved distal tip 110. In one embodiment, the heat setting procedure involves heating the FEP material to about (300-600)° F. for up to about 15 minutes.

In another example, a standard aluminum forming plate (not shown) having a channel taking the shape of the desired curvature is utilized. Because FEP is soft at room temperature, the FEP linear tube 130 renders the tube 130 flexible enough to be forced within the channel of the aluminum forming plate. Conductive heating elements raise the surface temperature of the channel, thereby heating the FEP linear tube 130 at a predetermined heating rate readily known to those of ordinary skill in the art. The linear tube 130 is heated until it becomes malleable and attains the shape of the curved channel. The residual stresses imparted to the linear FEP tube 130 when fitting the tube 130 into the curved channel at room temperature disappears upon the heat treatment. The permanently curved tube 130 is now quenched to room temperature by placing the aluminum forming plate on a cooling plate having chilled water running through tubes contained within the cooling plate. In one embodiment, the cool down cycle involves cooling the FEP material to about ambient temperature for about 5 minutes.

Once the linear FEP tube 110 has been transformed into a pre-curved distal tip 110, the curved tubular FEP member 150 may now be affixed to a non FEP proximal portion (e.g., PTFE) by a standard heat bond. No adhesive is required. Other methods for bonding and/or affixing the curved tubular FEP member 130 to a non FEP proximal portion will be apparent to those of ordinary skill in the art. Alternatively, the entire tubular shaft 130 may be formed from FEP. Although the heat setting techniques for imparting a curved orientation have been described in conjunction with mandrels and forming plates, other types of scaffolding structure may be used as known in the art. For example, heat setting with the use of a forming wire may be used to create the pre-curved distal tip 110.

The degree of curvature of the distal tip 110 can be characterized by an “angle of curvature”, which refers to the angle of the curved portion of the tubular member 130, in its relaxed state, as measured from a plane perpendicular to the longitudinal shaft of the tubular member 130 to the distal-most edge 161 of the tubular member 130. FIG. 1 shows that the angle of curvature is about 180 degrees. Other angles of curvature are contemplated, partially dependent upon the specific application. Additionally, the distal tip 110 may be characterized by a centerline diameter. The centerline diameter is defined as the diameter that the curved portion of the catheter, in its relaxed state, would create were it a full circle. It may span a range of several millimeters. Accordingly, the tightness of the curved configuration of the distal tip 110 is attributed to the angle of curvature and the centerline diameter.

Assembly of the sphincterotome 100 is as follows. As already mentioned, the FEP tubular member 130 is extruded by conventional extruding techniques and thereafter curvature is imparted to the extruded FEP member 130 as described above. The tubular member 130 is preferably formed with two lumens 170 and 210, a cutting wire lumen 170 and a wire guide lumen 210. More lumens may be utilized. Electrical conductor wire 200 is threaded through lumen 170. The cutting wire 160 may be formed by passing one free end of the electrical conductor wire 200 through opening 111 (FIG. 1) located in the wall of the distal region 140 of tubular member 130 and radially outward of the lumen 170. A radially bowed shape cutting wire 160 is formed when the distal end of the cutting wire 160 is reinserted into opening 112 of the wall of tubular member 130 and into lumen 170 where it is threaded proximally back therethrough and then secured by any means as known in the art within the lumen 170. The proximal end of conductor wire 200 is attached to electrical connector 180 and control handle 120.

FIG. 7 shows that packaging tray 710 may be utilized during the shipping and handling of the sphincterotome 100. A wire guide, such as loop tip wire guide 190 may be preloaded into wire guide lumen 210 (FIG. 2). The packaging tray 710 has a channel 760 that conforms to the natural curvature of pre-curved distal tip 110. In particular, channel 760 is designed to conform to pre-curved distal tip 110 and is configured with the same angle of curvature and centerline diameter created during imparting curvature to the initially straight extruded FEP tubular member 130, as explained above. The loop tip 191 of wire guide 190 preferably extends past the distal edge 161 (FIG. 1) of pre-curved distal tip 110 and is packaged within channel 761. Channel 761 is designed to conform to the loop shape of loop tip 191. Packaging tray 710 further includes handle opening 734 (FIG. 7) for housing control handle 120 (FIG. 1). FEP tubular member 130 is housed within channel 735. Another tray (not shown) compliments and mates with packaging tray 710 to enclose the sphincterotome 100. Accordingly, the packaging tray 710 provides an efficient way for accommodating the sphincterotome 100 with preloaded loop tip wire guide 190 or other device inserted therein during shipping, handling, and storage.

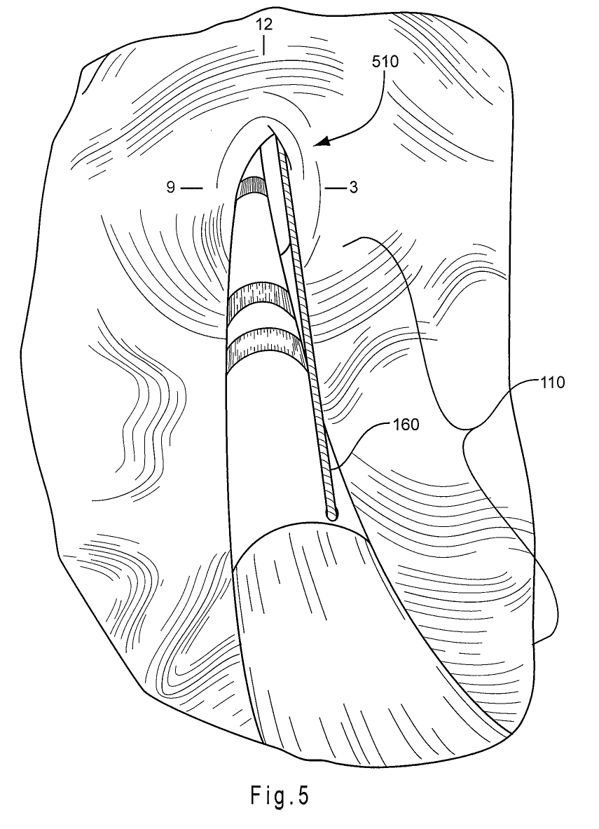

FIGS. 3, 5, and 6 illustrate how the sphincterotome 100 is used. The distal region 140 of tubular member 130 of sphincterotome 100 is preferably advanced within an accessory channel 380 of an endoscope 370 (FIG. 3). During advancement within the accessory channel 380, the pre-curved distal tip 110 flexes into a semi-straightened shape. If the loop tip wire guide 190 has been pre-loaded into the sphincterotome 100, then the tubular member 130 and the loop tip wire guide 190 are advanced simultaneously through the endoscope 370 until the distal tip 110 emerges from the distal opening of the accessory channel 380. As the distal tip 110 emerges from the distal end of the accessory channel 380, it relaxes back to its pre-curved shape. During an endoscopic sphinctertomy, the pre-curved distal tip 110 is bent at about 90 degrees or more as it emerges from the distal end of the accessory channel 380. The progression of the pre-curved distal tip 110 through the distal opening of the accessory channel 380 causes the tip 110 to orient automatically into the 12 o'clock position relative to the papilla 510 as shown in FIGS. 5 and 6. Radiopaque marker bands 165 (FIG. 6) along pre-curved distal tip 110 help the practitioner visualize the orientation and location of the pre-curved distal tip 110 relative to the papillary orifice 510. The 12 o'clock position is shown as a clock face about the papilla 510 and is illustrated in FIG. 5. The 12 o'clock position is preferable because it is most visible through the endoscope and it avoids injury to the duodenal wall. FIG. 5 shows the distal-most portion of distal tip 110 being advanced through the papilla 510 during cannulation of the biliary tree in the 12 o'clock position relative to the papillary orifice 510. The distal tip 110 is typically advanced along the loop tip wire guide 190, which is first advanced through the papilla 510 and is subsequently used for guiding the advancement of the sphincterotome 100.

Control handle 120 (FIG. 1) is proximally retracted to tighten cutting wire 160 to a flexed orientation as shown in FIG. 6. Cutting wire 160 is electrically energized via electrical conductor wire 200 to cut the center of the papilla 510. Manipulation of control handle 120 causes the cutting wire 160 to move into cutting plane P (FIG. 4) to cut papilla 510. The pre-curved distal tip 110 enables the cutting wire 160 to maintain orientation within cutting plane P as shown in FIG. 4. FIG. 4 shows that distal region 140 of tubular member 130 and cutting wire 160 is symmetrically disposed about the cutting plane P.

Performing the above described procedure with sphincterotome 100 is advantageous compared to using a normal sphincterotome for several reasons. Cannulating the biliary duct may become easier because the sphincterotome 100 has the capability to automatically steer and orient itself into the proper configuration during the sphincterotomy procedure. On the contrary, conventional sphincterotomes may sometimes require that a practitioner bend the distal end of the sphincterotome into the optimal orientation, which may require several iterations and often fails to retain its desired shape. The ability to more easily cannulate the duct with a FEP-formed sphincterotome 100 may also translate into reduced patient trauma because the cannulation may likely be achieved more quickly and/or accurately. Additionally, the elimination of a forming wire to maintain an unnatural curvature of conventional sphincterotomes formed from PTFE materials represents a cost reduction. The elimination of the forming wire also provides the opportunity to preload the lumen of the sphincterotome 100 with medical devices, such as loop tip wire guides 190. Additionally, because the forming wire merely maintains PTFE and other non-thermoplastic sphincterotomes in an unnatural curved position during shipping and handling, the sphincterotome upon use may revert back, to a certain degree, to its straightened configuration, thereby making endoscopic sphincterotomy difficult. The likelihood of reverting back to a straightened configuration may increase if the sphincterotome is not used for a prolonged period of time after shipment.

The above figures and disclosure are intended to be illustrative and not exhaustive. This description will suggest many variations and alternatives to one of ordinary skill in the art. All such variations and alternatives are intended to be encompassed within the scope of the attached claims. Those familiar with the art may recognize other equivalents to the specific embodiments described herein which equivalents are also intended to be encompassed by the attached claims.

1. An electrosurgical cutting device comprising:
a tubular member comprising a proximal portion and a distal end portion, the distal end portion comprising a heat-set, pre-curved distal tip formed from fluorinated ethylene propylene (FEP);
an electrically conductive cutting element located along the distal end portion of the tubular member, the cutting element connected to an electrical conductor extending within a lumen, the cutting element extending exteriorly of the tubular member along an inner radius of curvature of the distal tip, the cutting element moveable within a cutting plane; and
a wire guide lumen extending through at least a portion of the tubular member.

2. The cutting device of claim 1, further comprising a wire guide having a distal end, wherein the wire guide is disposed through the wire guide lumen such that the distal end of the wire guide extends beyond the distal dip.

3. The cutting device of claim 1, wherein the wire guide is a loop tip wire guide.

4. The electrosurgical device of claim 1, wherein the pre-curved distal tip is configured in a substantially linear orientation within the working channel.

5. The electrosurgical cutting device of claim 1, further comprising a kit, the kit including a packaging tray for the tubular member and the wire guide, the packaging tray including a curved channel corresponding to the pre-curved distal tip for retaining the heat-set, pre-curved distal tip therewithin.

6. The electrosurgical cutting device of claim 5, wherein the kit is characterized by an absence of a curved retention means for maintaining the shape of the pre-curved distal tip.

7. The electrosurgical cutting device of claim 1, wherein the distal end portion comprises an atraumatic tapered end.

8. The electrosurgical device of claim 1, further comprising a radiopaque marker band that is thermally bonded about the distal end portion of the tubular member.

9. The electrosurgical device of claim 1, wherein the cutting plane is oriented about a sphincter of a patient in a 12 o'clock position relative to the sphincter.

10. The electrosurgical cutting device of claim 1, wherein the electrically conductive cutting element is moveable between a first position and a second position.

11. The electrosurgical cutting device of claim 1, wherein the tubular member and cutting element are symmetrically disposed within the cutting plane.

12. The electrosurgical cutting device of claim 1, wherein the heat-set, pre-curved distal tip is straightened within a working channel of an endoscope.

13. The electrosurgical cutting device of claim 12, wherein the distal tip reverts to its pre-curved shape upon exiting the working channel.

14. The electrosurgical cutting device of claim 1, wherein the proximal portion is formed of PTFE, the proximal portion being heat bonded to the pre-curved distal tip.

15. A method of fabricating an electrosurgical cutting device, comprising the steps of:
(a) attaching a proximal end of an electrical conductor to an electrical connector of a handle, the handle being affixed to a substantially linear tubular member, the tubular member comprising a distal portion formed from fluorinated ethylene propylene (FEP);
(b) threading an electrical conductor through a lumen;
(c) passing a distal free end of the electrical conductor through a proximal luminal opening of the tubular member along a distal end of the distal portion and outward of the lumen to form a cutting element;
(d) reinserting a distal end of the cutting element through a distal luminal opening of the tubular member into the lumen and securing the distal end within the lumen; and
(e) heat setting the distal end of the distal end portion of the tubular member into a pre-curved shape that conforms to a shape of a scaffolding structure having a corresponding curved distal end.

16. The method of claim 15, wherein the heat setting step comprises:
placing a curved mandrel within a lumen of the FEP tubular member;
heating the FEP tubular member to a temperature sufficient for the member to attain the pre-curved shape corresponding to the curved distal end of the mandrel; and
cooling the FEP tubular member to solidify the pre-curved shape.

17. The method of claim 13, wherein the heat setting step comprises:
placing the tubular member within a mandrel having a curved distal end region;
heating the tubular member to a sufficient temperature so as to conform the distal end of the FEP tubular member with the curved distal end region of the mandrel; and
cooling the tubular member to solidify into a final shape having the pre-curved shape that substantially conforms to the curved distal end region of the mandrel.

18. The method of claim 15, further comprising the step of thermally bonding a radiopaque marker around the tubular member.

19. The method of claim 15, further comprising the step of shaping the distal portion of the tubular member into a tapered end.

20. The method of claim 15, further comprising the step of preloading a medical device through a lumen of the tubular member.

21. The method of claim 15, further comprising the step of preloading a loop tip wire guide through a wire guide lumen.

22. The method of claim 21, further comprising the step of packaging the device into a curved channel corresponding to the pre-curved shape.
